(19) United States
(12) Patent Application Publication
(10) Pub. No.: US 2019/0330306 A1
(43) Pub. Date: Oct. 31, 2019

(54) Compositions and Methods for Modulating the Immune System

(71) Applicant: WINDMIL THERAPEUTICS, INC., Baltimore, MD (US)

(72) Inventors: Kimberly A. Noonan, Baltimore, MD (US); Eric R. Lutz, Baltimore, MD (US); Ivan Borrello, Baltimore, MD (US)

(73) Assignee: WINDMIL THERAPEUTICS, INC., Baltimore, MD (US)

(21) Appl. No.: 16/471,854

(22) PCT Filed: Dec. 21, 2017
(86) PCT No.: PCT/US2017/067830

§ 371 Date: Jun. 20, 2019

Related U.S. Application Data
(60) Provisional application No. 62/438,106, filed on Dec. 22, 2016.

(51) Int. Cl. C07K 14/705; C12N 15/63

(52) U.S. Cl. CPC C07K 14/70578; C07K 14/70521; C07K 14/70596; C07K 2319/33; C12N 15/63; C07K 2319/03; C07K 2319/02; C07K 14/70517

(57) ABSTRACT

Embodiments provided for herein relate to compositions and methods related to chimeric proteins that can be used to modulate a subject's immune system and, for example, treat cancer.

### CROSS REFERENCE TO RELATED APPLICATIONS

This application claims priority to U.S. Provisional Application No. 62/438,106, filed Dec. 22, 2016, which is hereby incorporated by reference in its entirety.

### BACKGROUND

There are many compositions and methods to manipulate the immune system to treat cancer. However, there is still a need for improved compositions and methods to treat cancer. The presently disclosed subject matter fulfills these needs and others that would be evident to one of skill in the art

### SUMMARY

Embodiments disclosed herein provide for proteins comprising an extracellular domain of PD-1; a transmembrane domain selected from the group consisting of: 4-1BB transmembrane domain, CD28 transmembrane domain, CD27 transmembrane domain, and ICOS transmembrane domain; and an intracellular signaling domain selected from the group consisting of 4-1BB intracellular signaling domain, CD28 intracellular signaling domain, CD27 intracellular signaling domain, and ICOS intracellular signaling domain, and any combination thereof.

Embodiments disclosed herein also provide for nucleic acid molecules encoding the proteins provided herein.

In some embodiments, recombinant cells comprising the proteins or the nucleic acid molecules described herein are provided

In some embodiments, methods of making the recombinant cells are provided.

Embodiments for increasing an immune response or treating a neoplasm are also provided.

### DETAILED DESCRIPTION

As used herein, terms such as “a,” “an,” and “the” include singular and plural referents unless the context clearly demands otherwise.

As used in this document, terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used herein, the terms “treat,” “treated,” or “treating” mean both therapeutic treatment wherein the object is to slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Thus, “treatment of cancer” or “treating cancer” means an activity that alleviates or ameliorates any of the primary phenomena or secondary symptoms associated with the cancer or any other condition described herein. In some embodiments, the cancer that is being treated is one of the cancers recited herein.

As used herein, the term “autologous” can be used to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, multiple myeloma, lung cancer and the like. Examples of cancer also include but are not limited to, carcinoma, lymphoma, blastoma, sarcoma, and leukemia or lymphoid malignancies. More examples of such cancers include kidney or renal cancer, breast cancer, colon cancer, rectal cancer, colorectal cancer, lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, liver cancer, bladder cancer, cancer of the peritoneum, hepatocellular cancer, gastric or stomach cancer including gastrointestinal cancer, gastrointestinal stromal tumors (GIST), pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, salivary gland carcinoma, vulvar cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, urinary tract carcinomas, thyroid carcinomas, Wilm's tumor, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia, chronic myeloblastic leukemia; and post-transplant lymphoproliferative disorder (PTLD), as well as abnormal vascular proliferation associated with phakomatoses, edema (such as that associated with brain tumors), and Meigs' syndrome. “Tumor”, as used herein, refers to all neoplastic cell growth and proliferation, whether malignant or benign, and all pre-cancerous and cancerous cells and tissues.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

“Expression vector” refers to a vector comprising a recombinant polynucleotide comprising expression control sequences operatively linked to a nucleotide sequence to be expressed. An expression vector can comprise sufficient cis-acting elements for expression; other elements for expression can be supplied by the host cell or in an in vitro expression system. Expression vectors include all those known in the art, such as cosmids, plasmids (e g, naked or contained in liposomes) and viruses (e.g., lentiviruses, retroviruses, adenoviruses, and adeno-associated viruses) that incorporate the recombinant polynucleotide.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s). In some embodiments, the nucleotide sequence does not contain an intron and only contains a coding sequence.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses can achieve significant levels of gene transfer in vivo.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. In some embodiments, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCRTM, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various embodiments can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

A major hurdle in tumor immunology is the induction of tumor-specific tolerance which limits the intrinsic anti-tumor efficacy of many cell based approaches. Recent studies have shown significant clinical efficacy by targeting checkpoint inhibitors leading to the approval of anti-CTLA-4 and anti-PD-1 for metastatic melanoma. In some aspects, the embodiments relate to a chimeric receptor, comprising an extracellular domain expressing of a domain of a protein that can prevent the deactivation of the immune system and an activating intracellular domain. This has the advantage of hijacking the tolerogenic mechanisms into activating signals. This approach can be used in all clinical situations in which T cell anergy is a major aspect of the pathogenesis of the disease and where the antigen specificity is provided by the endogenous T cell repertoire.

In some aspects, the embodiments relate to a chimeric transmembrane protein, comprising an extracellular domain of an inhibitory receptor, a transmembrane domain, and an intracellular signaling domain. In some embodiments, the intracellular signaling domain can activate an immune response. The intracellular signaling domain may comprise a portion of an intracellular signaling protein. In some embodiments, the intracellular domain can be used to maintain the activation of a cell, such as a T-cell.

In some embodiments, the extracellular domain can transduce a signal to the intracellular signaling domain. For example, the extracellular domain may transduce a signal to the intracellular signaling domain upon binding an agonist of the native inhibitory receptor.

Signal transduction may comprise oligomerization of the protein. Oligomerization may comprise homo-oligomerization or hetero-oligomerization. Oligomerization may comprise dimerization of the protein, i.e., homo-dimerization with a second chimeric transmembrane protein or hetero-dimerization with a different protein.

Signal transduction may comprise phosphorylation. For example, the intracellular signaling domain may comprise kinase activity and/or a phosphorylation site. Signal transduction may comprise autophosphorylation, e.g., autophosphorylation of the intracellular signaling domain.

In some embodiments, the receptor, which can also be referred to as a “Switch Receptor” comprises an amino acid sequence described herein. In some embodiments, the receptor is encoded by a nucleic acid sequence described herein.

In some embodiments, the receptor comprises a CD8 Leader Peptide, a PD-1 Extracellular Domain, a 4-1BB Transmembrane Domain, and a 4-1BB Intracellular Domain. In some embodiments, the leader peptide is cleaved during the processing of the protein in a cell leaving a receptor comprising the PD-1 Extracellular Domain, a 4-1BB Transmembrane Domain, and a 4-1BB Intracellular Domain. In some embodiments, the domains comprise an amino acid sequence as described herein and below.

In some embodiments, the receptor comprises a CD8 Leader Peptide, a PD-1 Extracellular Domain, a CD28 Transmembrane Domain, and a CD28 Intracellular Domain. In some embodiments, the leader peptide is cleaved during the processing of the protein in a cell leaving a receptor comprising the PD-1 Extracellular Domain, a CD28 Transmembrane Domain, and a CD28 Intracellular Domain. In some embodiments, the domains comprise an amino acid sequence as described herein and below.

In some embodiments, the receptor comprises a CD8 Leader Peptide, a PD-1 Extracellular Domain, a CD28Transmembrane Domain, a CD28Intracellular Domain, and a 4-1BB Intracellular Domain. In some embodiments, the leader peptide is cleaved during the processing of the protein in a cell leaving a receptor comprising the PD-1 Extracellular Domain, a CD28Transmembrane Domain, a CD28 Intracellular Domain, and a 4-1BB Intracellular Domain. In some embodiments, the domains comprise an amino acid sequence as described herein and below.

In some embodiments, the receptor comprises a CD8 Leader Peptide, a PD-1 Extracellular Domain, a CD27 Transmembrane Domain, and a CD27 Intracellular Domain. In some embodiments, the leader peptide is cleaved during the processing of the protein in a cell leaving a receptor comprising the PD-1 Extracellular Domain, a CD27 Transmembrane Domain, and a CD27 Intracellular Domain. In some embodiments, the domains comprise an amino acid sequence as described herein and below.

In some embodiments, the receptor comprises a CD8 Leader Peptide, a PD-1 Extracellular Domain, a CD27 Transmembrane Domain, a CD27 Intracellular Domain, and a 4-1BB Intracellular Domain. In some embodiments, the leader peptide is cleaved during the processing of the protein in a cell leaving a receptor comprising the PD-1 Extracellular Domain, a CD27 Transmembrane Domain, a CD27 Intracellular Domain, and a 4-1BB Intracellular Domain. In some embodiments, the domains comprise an amino acid sequence as described herein and below.

In some embodiments, the receptor comprises a CD8 Leader Peptide, a PD-1 Extracellular Domain, a ICOS Transmembrane Domain, and a ICOS Intracellular Domain. In some embodiments, the leader peptide is cleaved during the processing of the protein in a cell leaving a receptor comprising the PD-1 Extracellular Domain, a ICOS Transmembrane Domain, and a ICOS Intracellular Domain. In some embodiments, the domains comprise an amino acid sequence as described herein and below.

In some embodiments, the extracellular domain is the extracellular domain of an inhibitory receptor. In some embodiments, the extracellular domain comprises a ligand-binding domain, e.g., the agonist-binding domain of the inhibitory receptor. In some embodiments, the extracellular domain comprises sufficient structure to transduce a signal across the membrane in response to ligand binding. Without being bound to any particular theory, for inhibitory receptors that transduce a signal by oligomerization mediated by a multivalent ligand, the mere presence of a ligand-binding domain may be sufficient structure to transduce a signal across the membrane in response to ligand binding. Without being bound to any particular theory, for inhibitory receptors that transduce a signal by altering the orientation of a transmembrane domain relative to the cell membrane, the extracellular domain may require native structure between the ligand-binding domain and transmembrane domain to transduce a signal across the membrane in response to ligand binding. For example, an extracellular domain may comprise the native sequence of the inhibitory receptor from its ligand-binding domain to its transmembrane domain.

The native inhibitory receptor can be a human inhibitory receptor or a mouse inhibitory receptor. Thus, the extracellular domain may comprise a human or mouse amino acid sequence. In some embodiments, the origin of the native inhibitory receptor is selected to match the species of a subject that is being treated, e.g., to avoid an immune response against the chimeric transmembrane protein. Nevertheless, the native inhibitory receptor may be selected from a different species, e.g., for convenience. Accordingly, the chimeric protein may or may not be xenogeneic-derived relative either to the species of cell in which the protein is expressed or the subject to which the protein is administered.

In some embodiments, the native inhibitory receptor is selected from proteins that reduce immune activity upon binding a native agonist. For example, the native inhibitory receptor may reduce T cell proliferation, T cell survival, cytokine secretion, or immune cytolytic activity upon binding a native agonist. The native inhibitory receptor may be a lymphocyte inhibitory receptor (i.e., the inhibitory receptor may be expressed on lymphocytes, such as T cells). For example, the native inhibitory receptor may be expressed on T cells, and the binding of an agonist to the native inhibitory receptor may cause cell signaling that disfavors T cell proliferation, T cell survival, cytokine secretion, or immune cytolytic activity.

In some embodiments, the native inhibitory receptor may be CTLA-4 (cytotoxic T-lymphocyte-associated protein 4; CD152), PD-1 (Programmed cell death protein 1; CD279), LAG-3 (Lymphocyte-activation gene 3; CD223), or Tim-3 (T cell immunoglobulin mucin-3). Thus, in some embodiments, the extracellular domain may be the extracellular domain from CTLA-4, PD-1, LAG-3, or Tim-3. The inhibitory receptor may be PD-1. In some embodiments, the transmembrane protein comprises the extracellular domain of PD-1. In some embodiments, the sequence of the extracellular domain comprises the PD-1 domain as described herein.

In some embodiments, the intracellular signaling domain is the signaling domain of an intracellular signaling protein. In some embodiments, the intracellular signaling domain may comprise kinase activity or a phosphorylation site. The intracellular signaling domain can, in some embodiments, activate a signaling molecule, such as a kinase or phosphorylase, e.g., following signal transduction across a cell membrane. The intracellular signaling domain may signal through a downstream kinase or a phosphorylase.

The intracellular signaling protein may be a human protein or a mouse protein. Thus, the intracellular signaling domain may comprise a human or mouse amino acid sequence. In some embodiments, the intracellular signaling protein is selected to match the species of a subject and cell that is being used for treatment, e.g., so that the signaling domain may utilize the cell's cytosolic machinery to activate downstream signaling molecules. Nevertheless, the intracellular signaling protein may be selected from a different species, e.g., for convenience, such as described above.

In some embodiments, the intracellular signaling protein increases immune activity. Thus, signal transduction via the chimeric transmembrane protein can result in a signal cascade that increases immune activity, wherein the intracellular signaling domain mediates the intracellular signaling cascade. In some embodiments, the intracellular signaling protein can enhance T cell proliferation, T cell survival, cytokine secretion, or immune cytolytic activity. In some embodiments, the intracellular signaling protein is a transmembrane protein or the intracellular signaling protein can bind a native transmembrane protein. The intracellular signaling protein may be a lymphocyte protein (i.e., the intracellular signaling protein may be expressed on lymphocytes, such as T cells).

In some embodiments, the intracellular signaling protein is CD3ζ (T-cell surface glycoprotein CD3 zeta chain; CD247), 4-1BB (tumor necrosis factor receptor superfamily member 9; CD137), or CD28 (T-cell-specific surface glycoprotein CD28; Tp44). Thus, the intracellular signaling protein may comprise a signaling domain from CD3ζ, 4-1BB, or CD28. The intracellular signaling protein may be 4-1BB. Thus, the intracellular signaling protein may comprise a signaling domain from 4-1BB. In some embodiments, the intracellular domain comprises the intracellular domains described herein.

In some embodiments, the chimeric transmembrane protein comprises a suicide domain, i.e., to kill a recombinant cell comprising the protein. The suicide domain may comprise thymidine kinase activity or caspase activity. For example, the suicide domain may be a thymidine kinase or a caspase. In some embodiments, the suicide domain is the thymidine kinase domain of HSV thymidine kinase (“HSV-TK”) or the suicide domain comprises a portion of caspase 9.

| | Amino Acid | |
|---|---|---|
| Sequence Name | Sequence | Nucleotide Sequence |
| CD Leader | MALPVTALLL | ATGGCCCTGCCCGTGACCGCCCTGCTCCTGCCTCTGGCTCTGCTGCTGCA |
| Peptide | PLALLLHAAR | TGCCGCCAGACCT (SEQ ID NO: 2) |
| | P (SEQ ID | |
| | NO: 1) | |
| PD-1 | PGWFLDSPDR | CCCGGCTGGTTCCTGGACAGCCCCGACAGACCCTGGAACCCTCCCACCTT |
| Extracellular | PWNPPTFSPA | CAGCCCTGCCCTGCTCGTGGTGACAGAGGGCGACAACGCCACCTTCACCT |
| Domain | LLVVTEGDNA | GTAGCTTCAGCAACACCAGCGAGAGCTTCGTGCTGAACTGGTACAGAATG |
| | TFTCSFSNTS | AGCCCCAGCAACCAGACCGACAAGCTGGCCGCCTTCCCCGAGGACAGAAG |
| | ESFVLNWYRM | CCAGCCCGGCCAGGACTGCCGGTTCAGAGTGACCCAGCTGCCCAACGGCC |
| | SPSNQTDKLA | GGGACTTCCACATGAGCGTGGTGCGCGCCAGACGGAACGACAGCGGCACA |
| | AFPEDRSQPG | TACCTGTGCGGCGCCATCAGCCTGGCCCCTAAGGCCCAGATCAAAGAGAG |
| | QDCRFRVTQL | CCTGCGGGCCGAGCTGAGAGTGACCGAGAGAAGGGCCGAGGTGCCCACCG |
| | PNGRDFHMSV | CCCACCCTAGCCCATCTCCAAGACCTGCCGGCCAGTTCCAGACCCTGGTG |
| | VRARRNDSGT | (SEQ ID NO: 4) |
| | YLCGAISLAP | |
| | KAQIKESLRA | |
| | ELRVTERRAE | |
| | VPTAHPSPSP | |
| | RPAGQFQTLV | |
| | (SEQ ID | |
| | NO: 3) | |
| 4-1BB | IISFFLALTS | ATCATCTCATTCTTTCTGGCCCTGACCAGCACAGCCCTGCTGTTTCTGCT |
| Transmembrane | TALLFLLFFL | GTTCTTCCTGACCCTGCGGTTCAGCGTGGTG (SEQ ID NO: 6) |
| Domain | TLRFSVV | |
| | (SEQ ID NO: | |
| | 5) | |
| 4-1BB | KRGRKKLLYI | AAACGGGGCAGAAAGAAGCTGCTGTACATCTTCAAGCAGCCCTTCATGCG |
| Intracellular | FKQPFMRPVQ | G (SEQ ID NO: 8) |
| Domain | TTQEEDGCSC | |
| | RFPEEEEGGC | |
| | EL (SEQ ID | |
| | NO: 7) | |
| CD28 | FWVLVVVGGV | TTTTGGGTGCTCGTGGTCGTTGGCGGAGTGCTGGCCTGTTATAGCCTGCT |
| Transmembrane | LACYSLLVTV | GGTCACCGTGGCCTTCATCATCTTTTGGGTC (SEQ ID NO: 10) |
| Domain | AFIIFWV | |
| | (SEQ ID | |
| | NO: 9) | |
| CD28 | RSKRSRLLHS | CGAAGCAAGCGGAGCCGGCTGCTGCACAGCGACTACATGAACATGACCCC |
| Intracellular | DYMNMTPRRP | TAGACGGCCCGGACCTACCAGAAAGCACTACCAGCCTTACGCTCCTCCTA |
| Domain | GPTRKHYQPY | GAGACTTCGCCGCCTACAGATCT (SEQ ID NO: 12) |
| | APPRDFAAYR | |
| | S (SEQ ID | |
| | NO: 11) | |
| CD27 | ILVIFSGMFL | ATCCTCGTGATCTTCAGCGGCATGTTCCTGGTGTTCACACTGGCTGGCGC |
| Transmembrane | VFTLAGALFL | CCTGTTTCTGCAC (SEQ ID NO: 14) |
| Domain | H (SEQ ID | |
| | NO: 13) | |
| CD27 | QRRKYRSNKG | CAGCGGAGAAAGTACAGAAGCAACAAGGGCGAGAGCCCCGTGGAACCTGC |
| Intracellular | ESPVEPAEPC | CGAGCCTTGTAGATACAGCTGTCCCAGAGAGGAAGAGGGCAGCACAATCC |
| Domain | RYSCPREEEG | CCATCCAAGAGGACTACAGAAAGCCCGAGCCTGCCTGCTCTCCT (SEQ |
| | STIPIQEDYR | ID NO: 16) |
| | KPEPACSP | |
| | (SEQ ID | |
| | NO: 15) | |
| ICOS | FWLPIGCAAF | TTCTGGCTGCCTATCGGCTGTGCCGCTTTTGTGGTCGTGTGCATCCTGGG |
| Transmembrane | VVVCILGCIL | CTGCATCCTGATC (SEQ ID NO: 18) |
| Domain | I (SEQ ID | |
| | NO: 17) | |
| ICOS | CWLTKKKYSS | TGCTGGCTGACCAAGAAAAAGTACAGCAGCAGCGTGCACGACCCCAACGG |
| Intracellular | SVHDPNGEYM | CGAGTACATGTTCATGAGAGCCGTGAACACCGCCAAGAAGTCCAGACTGA |
| Domain | FMRAVNTAKK | CCGACGTGACCCTT (SEQ ID NO: 20) |
| | SRLTDVTL | |
| | (SEQ ID | |
| | NO: 19) | |

In some embodiments, the switch receptor comprises a sequence provided for in the table below:

| Receptor | | |
|---|---|---|
| Domains | Amino Acid Sequence | Nucleotide Sequence. |
| PD-1 | PGWFLDSPDRPWNPPTFSPA | CCCGGCTGGTTCCTGGACAGCCCCGACAGACCCTGGAAC |
| Extracellular | LLVVTEGDNATFTCSFSNTS | CCTCCCACCTTCAGCCCTGCCCTGCTCGTGGTGACAGAG |
| Domain; 4-1BB | ESFVLNWYRMSPSNQTDKLA | GGCGACAACGCCACCTTCACCTGTAGCTTCAGCAACACC |
| Transmembrane | AFPEDRSQPGQDCRFRVTQL | AGCGAGAGCTTCGTGCTGAACTGGTACAGAATGAGCCCC |
| Domain; 4-1BB | PNGRDFHMSVVRARRNDSGT | AGCAACCAGACCGACAAGCTGGCCGCCTTCCCCGAGGAC |
| Intracellular | YLCGAISLAPKAQIKESLRA | AGAAGCCAGCCCGGCCAGGACTGCCGGTTCAGAGTGACC |
| Domain | ELRVTERRAEVPTAHPSPSP | CAGCTGCCCAACGGCCGGGACTTCCACATGAGCGTGGTG |
| | RPAGQFQTLVIISFFLALTS | CGCGCCAGACGGAACGACAGCGGCACATACCTGTGCGGC |
| | TALLFLLFFLTLRFSVVKRG | GCCATCAGCCTGGCCCCTAAGGCCCAGATCAAAGAGAGC |
| | RKKLLYIFKQPFMRPVQTTQ | CTGCGGGCCGAGCTGAGAGTGACCGAGAGAAGGGCCGAG |
| | EEDGCSCRFPEEEEGGCEL | GTGCCCACCGCCCACCCTAGCCCATCTCCAAGACCTGCC |
| | (SEQ ID NO: 21) | GGCCAGTTCCAGACCCTGGTGATCATCTCATTCTTTCTG |
| | | GCCCTGACCAGCACAGCCCTGCTGTTTCTGCTGTTCTTC |
| | | CTGACCCTGCGGTTCAGCGTGGTGAAACGGGGCAGAAAG |
| | | AAGCTGCTGTACATCTTCAAGCAGCCCTTCATGCGGCCC |
| | | GTGCAGACCACCCAGGAAGAAGACGGCTGCAGCTGCCGG |
| | | TTCCCCGAAGAAGAAGAGGGCGGCTGCGAACTG (SEQ |
| | | ID NO: 22) |
| PD-1 | PGWFLDSPDRPWNPPTFSPA | CCAGGCTGGTTCCTGGACTCTCCCGACAGACCTTGGAAC |
| Extracellular | LLVVTEGDNATFTCSFSNTS | CCTCCAACATTCAGCCCCGCTCTGCTGGTGGTTACCGAG |
| Domain; | ESFVLNWYRMSPSNQTDKLA | GGCGATAATGCCACCTTCACCTGTAGCTTCAGCAACACC |
| CD28 | AFPEDRSQPGQDCRFRVTQL | AGCGAGAGCTTCGTGCTGAACTGGTACAGAATGAGCCCC |
| Transmembrane | PNGRDFHMSVVRARRNDSGT | AGCAACCAGACCGACAAGCTGGCCGCCTTTCCTGAGGAT |
| Domain; | YLCGAISLAPKAQIKESLRA | AGATCTCAGCCCGGCCAGGACTGCCGGTTCAGAGTTACA |
| CD28 | ELRVTERRAEVPTAHPSPSP | CAGCTGCCCAACGGCCGGGACTTCCACATGTCTGTCGTC |
| Intracellular | RPAGQFQTLVFWVLVVVGGV | CGGGCCAGAAGAAACGACAGCGGCACATATCTGTGCGGC |
| Domain | LACYSLLVTVAFIIFWVRSK | GCCATTTCTCTGGCCCCTAAGGCTCAGATCAAAGAGAGC |
| | RSRLLHSDYMNMTPRRPGPT | CTGAGAGCCGAGCTGAGAGTGACAGAAAGACGGGCCGAA |
| | RKHYQPYAPPRDFAAYRS | GTGCCCACAGCTCACCCTTCACCTTCTCCAAGACCTGCC |
| | (SEQ ID NO: 23) | GGCCAGTTTCAGACCCTGGTGTTTTGGGTGCTCGTGGTC |
| | | GTTGGCGGAGTGCTGGCCTGTTATAGCCTGCTGGTCACC |
| | | GTGGCCTTCATCATCTTTTGGGTCCGAAGCAAGCGGAGC |
| | | CGGCTGCTGCACAGCGACTACATGAACATGACCCCTAGA |
| | | CGGCCCGGACCTACCAGAAAGCACTACCAGCCTTACGCT |
| | | CCTCCTAGAGACTTCGCCGCCTACAGATCT (SEQ ID |
| | | NO: 24) |
| PD-1 | PGWFLDSPDRPWNPPTFSPA | CCAGGCTGGTTCCTGGACTCTCCCGACAGACCTTGGAAC |
| Extracellular | LLVVTEGDNATFTCSFSNTS | CCTCCAACATTCAGCCCCGCTCTGCTGGTGGTTACCGAG |
| Domain; | ESFVLNWYRMSPSNQTDKLA | GGCGATAATGCCACCTTCACCTGTAGCTTCAGCAACACC |
| CD28 | AFPEDRSQPGQDCRFRVTQL | AGCGAGAGCTTCGTGCTGAACTGGTACAGAATGAGCCCC |
| Transmembrane | PNGRDFHMSVVRARRNDSGT | AGCAACCAGACCGACAAGCTGGCCGCCTTTCCTGAGGAT |
| Domain; | YLCGAISLAPKAQIKESLRA | AGATCTCAGCCCGGCCAGGACTGCCGGTTCAGAGTTACA |
| CD28 | ELRVTERRAEVPTAHPSPSP | CAGCTGCCCAACGGCCGGGACTTCCACATGTCTGTCGTC |
| Intracellular | RPAGQFQTLVFWVLVVVGGV | CGGGCCAGAAGAAACGACAGCGGCACATATCTGTGCGGC |
| Domain; | LACYSLLVTVAFIIFWVRSK | GCCATTTCTCTGGCCCCTAAGGCTCAGATCAAAGAGAGC |
| 4-1BB | RSRLLHSDYMNMTPRRPGPT | CTGAGAGCCGAGCTGAGAGTGACAGAAAGACGGGCCGAA |
| Intracellular | RKHYQPYAPPRDFAAYRSKR | GTGCCCACAGCTCACCCTTCACCTTCTCCAAGACCTGCC |
| Domain | GRKKLLYIFKQPFMRPVQTT | GGCCAGTTTCAGACCCTGGTGTTTTGGGTGCTCGTGGTC |
| | QEEDGCSCRFPEEEEGGCEL | GTTGGCGGAGTGCTGGCCTGTTATAGCCTGCTGGTCACC |
| | (SEQ ID NO: 25) | GTGGCCTTCATCATCTTTTGGGTCCGAAGCAAGCGGAGC |
| | | CGGCTGCTGCACAGCGACTACATGAACATGACCCCTAGA |
| | | CGGCCCGGACCTACCAGAAAGCACTACCAGCCTTACGCT |
| | | CCTCCTAGAGACTTCGCCGCCTACAGATCCAAGCGGGGC |
| | | AGAAAGAAGCTGCTGTACATCTTCAAGCAGCCCTTCATG |
| | | CGGCCCGTGCAGACCACACAAGAGGAAGATGGCTGCTCC |
| | | TGCAGATTCCCCGAGGAAGAAGAAGGCGGCTGCGAGCTT |
| | | (SEQ ID NO: 26) |
| PD-1 | PGWFLDSPDRPWNPPTFSPA | CCAGGCTGGTTCCTGGACTCTCCCGACAGACCTTGGAAC |
| Extracellular | LLVVTEGDNATFTCSFSNTS | CCTCCAACATTCAGCCCCGCTCTGCTGGTGGTTACCGAG |
| Domain; | ESFVLNWYRMSPSNQTDKLA | GGCGATAATGCCACCTTCACCTGTAGCTTCAGCAACACC |
| CD27 | AFPEDRSQPGQDCRFRVTQL | AGCGAGAGCTTCGTGCTGAACTGGTACAGAATGAGCCCC |
| Transmembrane | PNGRDFHMSVVRARRNDSGT | AGCAACCAGACCGACAAGCTGGCCGCCTTTCCTGAGGAT |
| Domain; | YLCGAISLAPKAQIKESLRA | AGATCTCAGCCCGGCCAGGACTGCCGGTTCAGAGTTACA |
| CD27 | ELRVTERRAEVPTAHPSPSP | CAGCTGCCCAACGGCCGGGACTTCCACATGTCTGTCGTC |
| Intracellular | RPAGQFQTLVILVIFSGMFL | CGGGCCAGAAGAAACGACAGCGGCACATATCTGTGCGGC |
| Domain | VFTLAGALFLHQRRKYRSNK | GCCATTTCTCTGGCCCCTAAGGCTCAGATCAAAGAGAGC |
| | GESPVEPAEPCRYSCPREEE | CTGAGAGCCGAGCTGAGAGTGACAGAAAGACGGGCCGAA |
| | GSTIPIQEDYRKPEPACSP | GTGCCCACAGCTCACCCTTCACCTTCTCCAAGACCTGCC |
| | (SEQ ID NO: 27) | GGCCAGTTTCAGACCCTGGTCATCCTCGTGATCTTCAGC |
| | | GGCATGTTCCTGGTGTTCACACTGGCTGGCGCCCTGTTT |
| | | CTGCACCAGCGGAGAAAGTACAGAAGCAACAAGGGCGAG |
| | | AGCCCCGTGGAACCTGCCGAGCCTTGTAGATACAGCTGT |
| | | CCCAGAGAGGAAGAGGGCAGCACAATCCCCATCCAAGAG |
| | | GACTACAGAAAGCCCGAGCCTGCCTGCTCTCCT (SEQ |
| | | ID NO: 28) |
| PD-1 | PGWFLDSPDRPWNPPTFSPA | CCAGGCTGGTTCCTGGACTCTCCCGACAGACCTTGGAAC |
| Extracellular | LLVVTEGDNATFTCSFSNTS | CCTCCAACATTCAGCCCCGCTCTGCTGGTGGTTACCGAG |
| Domain; | ESFVLNWYRMSPSNQTDKLA | GGCGATAATGCCACCTTCACCTGTAGCTTCAGCAACACC |
| CD27 | AFPEDRSQPGQDCRFRVTQL | AGCGAGAGCTTCGTGCTGAACTGGTACAGAATGAGCCCC |
| Transmembrane | PNGRDFHMSVVRARRNDSGT | AGCAACCAGACCGACAAGCTGGCCGCCTTTCCTGAGGAT |
| Domain; | YLCGAISLAPKAQIKESLRA | AGATCTCAGCCCGGCCAGGACTGCCGGTTCAGAGTTACA |
| CD27 | ELRVTERRAEVPTAHPSPSP | CAGCTGCCCAACGGCCGGGACTTCCACATGTCTGTCGTC |
| Intracellular | RPAGQFQTLVILVIFSGMFL | CGGGCCAGAAGAAACGACAGCGGCACATATCTGTGCGGC |
| Domain; | VFTLAGALFLHQRRKYRSNK | GCCATTTCTCTGGCCCCTAAGGCTCAGATCAAAGAGAGC |
| 4-1BB | GESPVEPAEPCRYSCPREEE | CTGAGAGCCGAGCTGAGAGTGACAGAAAGACGGGCCGAA |
| Intracellular | GSTIPIQEDYRKPEPACSPK | GTGCCCACAGCTCACCCTTCACCTTCTCCAAGACCTGCC |
| Domain | RGRKKLLYIFKQPFMRPVQT | GGCCAGTTTCAGACCCTGGTCATCCTCGTGATCTTCAGC |
| | TQEEDGCSCRFPEEEEGGCE | GGCATGTTCCTGGTGTTCACACTGGCTGGCGCCCTGTTT |
| | L (SEQ ID NO: 29) | CTGCACCAGCGGAGAAAGTACAGAAGCAACAAGGGCGAG |
| | | AGCCCCGTGGAACCTGCCGAGCCTTGTAGATACAGCTGT |
| | | CCCAGAGAGGAAGAGGGCAGCACAATCCCCATCCAAGAG |
| | | GACTACAGAAAGCCCGAGCCTGCCTGCTCTCCCAAGAGA |
| | | GGCAGAAAGAAGCTGCTGTACATCTTCAAGCAGCCCTTC |
| | | ATGCGGCCCGTGCAGACCACACAAGAGGAAGATGGCTGC |
| | | TCCTGCAGATTCCCCGAGGAAGAAGAAGGCGGCTGCGAG |
| | | CTT (SEQ ID NO: 30) |
| PD-1 | PGWFLDSPDRPWNPPTFSPA | CCAGGCTGGTTCCTGGACTCTCCCGACAGACCTTGGAAC |
| Extracellular | LLVVTEGDNATFTCSFSNTS | CCTCCAACATTCAGCCCCGCTCTGCTGGTGGTTACCGAG |
| Domain; | ESFVLNWYRMSPSNQTDKLA | GGCGATAATGCCACCTTCACCTGTAGCTTCAGCAACACC |
| ICOS | AFPEDRSQPGQDCRFRVTQL | AGCGAGAGCTTCGTGCTGAACTGGTACAGAATGAGCCCC |
| Transmembrane | PNGRDFHMSVVRARRNDSGT | AGCAACCAGACCGACAAGCTGGCCGCCTTTCCTGAGGAT |
| Domain; | YLCGAISLAPKAQIKESLRA | AGATCTCAGCCCGGCCAGGACTGCCGGTTCAGAGTTACA |
| ICOS | ELRVTERRAEVPTAHPSPSP | CAGCTGCCCAACGGCCGGGACTTCCACATGTCTGTCGTC |
| Intracellular | RPAGQFQTLVFWLPIGCAAF | CGGGCCAGAAGAAACGACAGCGGCACATATCTGTGCGGC |
| Domain | VVVCILGCILICWLTKKKYS | GCCATTTCTCTGGCCCCTAAGGCTCAGATCAAAGAGAGC |
| | SSVHDPNGEYMFMRAVNTAK | CTGAGAGCCGAGCTGAGAGTGACAGAAAGACGGGCCGAA |
| | KSRLTDVTL (SEQ ID | GTGCCCACAGCTCACCCTTCACCTTCTCCAAGACCTGCC |
| | NO: 31) | GGCCAGTTTCAGACCCTGGTGTTCTGGCTGCCTATCGGC |
| | | TGTGCCGCTTTTGTGGTCGTGTGCATCCTGGGCTGCATC |
| | | CTGATCTGCTGGCTGACCAAGAAAAAGTACAGCAGCAGC |
| | | GTGCACGACCCCAACGGCGAGTACATGTTCATGAGAGCC |
| | | GTGAACACCGCCAAGAAGTCCAGACTGACCGACGTGACC |
| | | CTT (SEQ ID NO: 32) |

In some embodiments, the receptors provided for in the table above comprise a N-terminal CD8 leader peptide sequence. In some embodiments, the leader sequence is MALPVTALLLPLALLLHAARP (SEQ ID NO: 1). If the receptor comprises the leader sequence it can be appended to the N-terminus of the sequence in the table and form a contiguous sequence. The leader sequence can be encoded, for example, a nucleotide sequence of SEQ ID NO: 2. The intracellular domains provided herein can also be referred to as intracellular signaling domains.

Accordingly, in some embodiments, the receptor comprises a sequence as provided in the following table:

| Receptor Domains | | |
|---|---|---|
| with CDB Leader | | |
| Sequence | Amino Acid Sequence | Nucleotide Sequence. |
| PD-1 | MALPVTALLLPLALLLHAAR | ATGGCCCTGCCCGTGACCGCCCTGCTCCTGCCTCTGGCT |
| Extracellular | PPGWFLDSPDRPWNPPTFSP | CTGCTGCTGCATGCCGCCAGACCTCCCGGCTGGTTCCTG |
| Domain; 4-1BB | ALLVVTEGDNATFTCSFSNT | GACAGCCCCGACAGACCCTGGAACCCTCCCACCTTCAGC |
| Transmembrane | SESFVLNWYRMSPSNQTDKL | CCTGCCCTGCTCGTGGTGACAGAGGGCGACAACGCCACC |
| Domain; 4-1BB | AAFPEDRSQPGQDCRFRVTQ | TTCACCTGTAGCTTCAGCAACACCAGCGAGAGCTTCGTG |
| Intracellular | LPNGRDFHMSVVRARRNDSG | CTGAACTGGTACAGAATGAGCCCCAGCAACCAGACCGAC |
| Domain | TYLCGAISLAPKAQIKESLR | AAGCTGGCCGCCTTCCCCGAGGACAGAAGCCAGCCCGGC |
| | AELRVTERRAEVPTAHPSPS | CAGGACTGCCGGTTCAGAGTGACCCAGCTGCCCAACGGC |
| | PRPAGQFQTLVIISFFLALT | CGGGACTTCCACATGAGCGTGGTGCGCGCCAGACGGAAC |
| | STALLFLLFFLTLRFSVVKR | GACAGCGGCACATACCTGTGCGGCGCCATCAGCCTGGCC |
| | GRKKLLYIFKQPFMRPVQTT | CCTAAGGCCCAGATCAAAGAGAGCCTGCGGGCCGAGCTG |
| | QEEDGCSCRFPEEEEGGCEL | AGAGTGACCGAGAGAAGGGCCGAGGTGCCCACCGCCCAC |
| | (SEQ ID NO: 33) | CCTAGCCCATCTCCAAGACCTGCCGGCCAGTTCCAGACC |
| | | CTGGTGATCATCTCATTCTTTCTGGCCCTGACCAGCACA |
| | | GCCCTGCTGTTTCTGCTGTTCTTCCTGACCCTGCGGTTC |
| | | AGCGTGGTGAAACGGGGCAGAAAGAAGCTGCTGTACATC |
| | | TTCAAGCAGCCCTTCATGCGGCCCGTGCAGACCACCCAG |
| | | GAAGAAGACGGCTGCAGCTGCCGGTTCCCCGAAGAAGAA |
| | | GAGGGCGGCTGCGAACTG (SEQ ID NO: 34) |
| PD-1 | MALPVTALLLPLALLLHAAR | ATGGCCCTGCCTGTGACAGCTCTGCTGCTGCCTCTTGCT |
| Extracellular | PPGWFLDSPDRPWNPPTFSP | CTGCTTCTGCATGCCGCTAGACCTCCAGGCTGGTTCCTG |
| Domain; | ALLVVTEGDNATFTCSFSNT | GACTCTCCCGACAGACCTTGGAACCCTCCAACATTCAGC |
| CD28 | SESFVLNWYRMSPSNQTDKL | CCCGCTCTGCTGGTGGTTACCGAGGGCGATAATGCCACC |
| Transmembrane | AAFPEDRSQPGQDCRFRVTQ | TTCACCTGTAGCTTCAGCAACACCAGCGAGAGCTTCGTG |
| Domain; | LPNGRDFHMSVVRARRNDSG | CTGAACTGGTACAGAATGAGCCCCAGCAACCAGACCGAC |
| CD28 | TYLCGAISLAPKAQIKESLR | AAGCTGGCCGCCTTTCCTGAGGATAGATCTCAGCCCGGC |
| Intracellular | AELRVTERRAEVPTAHPSPS | CAGGACTGCCGGTTCAGAGTTACACAGCTGCCCAACGGC |
| Domain | PRPAGQFQTLVFWVLVVVGG | CGGGACTTCCACATGTCTGTCGTCCGGGCCAGAAGAAAC |
| | VLACYSLLVTVAFIIFWVRS | GACAGCGGCACATATCTGTGCGGCGCCATTTCTCTGGCC |
| | KRSRLLHSDYMNMTPRRPGP | CCTAAGGCTCAGATCAAAGAGAGCCTGAGAGCCGAGCTG |
| | TRKHYQPYAPPRDFAAYRS | AGAGTGACAGAAAGACGGGCCGAAGTGCCCACAGCTCAC |
| | (SEQ ID NO: 35) | CCTTCACCTTCTCCAAGACCTGCCGGCCAGTTTCAGACC |
| | | CTGGTGTTTTGGGTGCTCGTGGTCGTTGGCGGAGTGCTG |
| | | GCCTGTTATAGCCTGCTGGTCACCGTGGCCTTCATCATC |
| | | TTTTGGGTCCGAAGCAAGCGGAGCCGGCTGCTGCACAGC |
| | | GACTACATGAACATGACCCCTAGACGGCCCGGACCTACC |
| | | AGAAAGCACTACCAGCCTTACGCTCCTCCTAGAGACTTC |
| | | GCCGCCTACAGATCT (SEQ ID NO: 36) |
| PD-1 | MALPVTALLLPLALLLHAAR | ATGGCCCTGCCTGTGACAGCTCTGCTGCTGCCTCTTGCT |
| Extracellular | PPGWFLDSPDRPWNPPTFSP | CTGCTTCTGCATGCCGCTAGACCTCCAGGCTGGTTCCTG |
| Domain; | ALLVVTEGDNATFTCSFSNT | GACTCTCCCGACAGACCTTGGAACCCTCCAACATTCAGC |
| CD28 | SESFVLNWYRMSPSNQTDKL | CCCGCTCTGCTGGTGGTTACCGAGGGCGATAATGCCACC |
| Transmembrane | AAFPEDRSQPGQDCRFRVTQ | TTCACCTGTAGCTTCAGCAACACCAGCGAGAGCTTCGTG |
| Domain; | LPNGRDFHMSVVRARRNDSG | CTGAACTGGTACAGAATGAGCCCCAGCAACCAGACCGAC |
| CD28 | TYLCGAISLAPKAQIKESLR | AAGCTGGCCGCCTTTCCTGAGGATAGATCTCAGCCCGGC |
| Intracellular | AELRVTERRAEVPTAHPSPS | CAGGACTGCCGGTTCAGAGTTACACAGCTGCCCAACGGC |
| Domain; | PRPAGQFQTLVFWVLVVVGG | CGGGACTTCCACATGTCTGTCGTCCGGGCCAGAAGAAAC |
| 4-1BB | VLACYSLLVTVAFIIFWVRS | GACAGCGGCACATATCTGTGCGGCGCCATTTCTCTGGCC |
| Intracellular | KRSRLLHSDYMNMTPRRPGP | CCTAAGGCTCAGATCAAAGAGAGCCTGAGAGCCGAGCTG |
| Domain | TRKHYQPYAPPRDFAAYRSK | AGAGTGACAGAAAGACGGGCCGAAGTGCCCACAGCTCAC |
| | RGRKKLLYIFKQPFMRPVQT | CCTTCACCTTCTCCAAGACCTGCCGGCCAGTTTCAGACC |
| | TQEEDGCSCRFPEEEEGGCE | CTGGTGTTTTGGGTGCTCGTGGTCGTTGGCGGAGTGCTG |
| | L (SEQ ID NO: 37) | GCCTGTTATAGCCTGCTGGTCACCGTGGCCTTCATCATC |
| | | TTTTGGGTCCGAAGCAAGCGGAGCCGGCTGCTGCACAGC |
| | | GACTACATGAACATGACCCCTAGACGGCCCGGACCTACC |
| | | AGAAAGCACTACCAGCCTTACGCTCCTCCTAGAGACTTC |
| | | GCCGCCTACAGATCCAAGCGGGGCAGAAAGAAGCTGCTG |
| | | TACATCTTCAAGCAGCCCTTCATGCGGCCCGTGCAGACC |
| | | ACACAAGAGGAAGATGGCTGCTCCTGCAGATTCCCCGAG |
| | | GAAGAAGAAGGCGGCTGCGAGCTT |
| | | (SEQ ID NO: 38) |
| PD-1 | MALPVTALLLPLALLLHAAR | ATGGCCCTGCCTGTGACAGCTCTGCTGCTGCCTCTTGCT |
| Extracellular | PPGWFLDSPDRPWNPPTFSP | CTGCTTCTGCATGCCGCTAGACCTCCAGGCTGGTTCCTG |
| Domain; | ALLVVTEGDNATFTCSFSNT | GACTCTCCCGACAGACCTTGGAACCCTCCAACATTCAGC |
| CD27 | SESFVLNWYRMSPSNQTDKL | CCCGCTCTGCTGGTGGTTACCGAGGGCGATAATGCCACC |
| Transmembrane | AAFPEDRSQPGQDCRFRVTQ | TTCACCTGTAGCTTCAGCAACACCAGCGAGAGCTTCGTG |
| Domain; | LPNGRDFHMSVVRARRNDSG | CTGAACTGGTACAGAATGAGCCCCAGCAACCAGACCGAC |
| CD27 | TYLCGAISLAPKAQIKESLR | AAGCTGGCCGCCTTTCCTGAGGATAGATCTCAGCCCGGC |
| Intracellular | AELRVTERRAEVPTAHPSPS | CAGGACTGCCGGTTCAGAGTTACACAGCTGCCCAACGGC |
| Domain | PRPAGQFQTLVILVIFSGMF | CGGGACTTCCACATGTCTGTCGTCCGGGCCAGAAGAAAC |
| | LVFTLAGALFLHQRRKYRSN | GACAGCGGCACATATCTGTGCGGCGCCATTTCTCTGGCC |
| | KGESPVEPAEPCRYSCPREE | CCTAAGGCTCAGATCAAAGAGAGCCTGAGAGCCGAGCTG |
| | EGSTIPIQEDYRKPEPACSP | AGAGTGACAGAAAGACGGGCCGAAGTGCCCACAGCTCAC |
| | (SEQ ID NO: 39) | CCTTCACCTTCTCCAAGACCTGCCGGCCAGTTTCAGACC |
| | | CTGGTCATCCTCGTGATCTTCAGCGGCATGTTCCTGGTG |
| | | TTCACACTGGCTGGCGCCCTGTTTCTGCACCAGCGGAGA |
| | | AAGTACAGAAGCAACAAGGGCGAGAGCCCCGTGGAACCT |
| | | GCCGAGCCTTGTAGATACAGCTGTCCCAGAGAGGAAGAG |
| | | GGCAGCACAATCCCCATCCAAGAGGACTACAGAAAGCCC |
| | | GAGCCTGCCTGCTCTCCT (SEQ ID NO: 40) |
| PD-1 | MALPVTALLLPLALLLHAAR | ATGGCCCTGCCTGTGACAGCTCTGCTGCTGCCTCTTGCT |
| Extracellular | PPGWFLDSPDRPWNPPTFSP | CTGCTTCTGCATGCCGCTAGACCTCCAGGCTGGTTCCTG |
| Domain; | ALLVVTEGDNATFTCSFSNT | GACTCTCCCGACAGACCTTGGAACCCTCCAACATTCAGC |
| CD27 | SESFVLNWYRMSPSNQTDKL | CCCGCTCTGCTGGTGGTTACCGAGGGCGATAATGCCACC |
| Transmembrane | AAFPEDRSQPGQDCRFRVTQ | TTCACCTGTAGCTTCAGCAACACCAGCGAGAGCTTCGTG |
| Domain; | LPNGRDFHMSVVRARRNDSG | CTGAACTGGTACAGAATGAGCCCCAGCAACCAGACCGAC |
| CD27 | TYLCGAISLAPKAQIKESLR | AAGCTGGCCGCCTTTCCTGAGGATAGATCTCAGCCCGGC |
| Intracellular | AELRVTERRAEVPTAHPSPS | CAGGACTGCCGGTTCAGAGTTACACAGCTGCCCAACGGC |
| Domain; | PRPAGQFQTLVILVIFSGMF | CGGGACTTCCACATGTCTGTCGTCCGGGCCAGAAGAAAC |
| 4-1BB | LVFTLAGALFLHQRRKYRSN | GACAGCGGCACATATCTGTGCGGCGCCATTTCTCTGGCC |
| Intracellular | KGESPVEPAEPCRYSCPREE | CCTAAGGCTCAGATCAAAGAGAGCCTGAGAGCCGAGCTG |
| Domain | EGSTIPIQEDYRKPEPACSP | AGAGTGACAGAAAGACGGGCCGAAGTGCCCACAGCTCAC |
| | KRGRKKLLYIFKQPFMRPVQ | CCTTCACCTTCTCCAAGACCTGCCGGCCAGTTTCAGACC |
| | TTQEEDGCSCRFPEEEEGGC | CTGGTCATCCTCGTGATCTTCAGCGGCATGTTCCTGGTG |
| | EL (SEQ ID NO: 41) | TTCACACTGGCTGGCGCCCTGTTTCTGCACCAGCGGAGA |
| | | AAGTACAGAAGCAACAAGGGCGAGAGCCCCGTGGAACCT |
| | | GCCGAGCCTTGTAGATACAGCTGTCCCAGAGAGGAAGAG |
| | | GGCAGCACAATCCCCATCCAAGAGGACTACAGAAAGCCC |
| | | GAGCCTGCCTGCTCTCCCAAGAGAGGCAGAAAGAAGCTG |
| | | CTGTACATCTTCAAGCAGCCCTTCATGCGGCCCGTGCAG |
| | | ACCACACAAGAGGAAGATGGCTGCTCCTGCAGATTCCCC |
| | | GAGGAAGAAGAAGGCGGCTGCGAGCTT (SEQ ID NO: |
| | | 42) |
| PD-1 | MALPVTALLLPLALLLHAAR | ATGGCCCTGCCTGTGACAGCTCTGCTGCTGCCTCTTGCT |
| Extracellular | PPGWFLDSPDRPWNPPTFSP | CTGCTTCTGCATGCCGCTAGACCTCCAGGCTGGTTCCTG |
| Domain; | ALLVVTEGDNATFTCSFSNT | GACTCTCCCGACAGACCTTGGAACCCTCCAACATTCAGC |
| ICOS | SESFVLNWYRMSPSNQTDKL | CCCGCTCTGCTGGTGGTTACCGAGGGCGATAATGCCACC |
| Transmembrane | AAFPEDRSQPGQDCRFRVTQ | TTCACCTGTAGCTTCAGCAACACCAGCGAGAGCTTCGTG |
| Domain; | LPNGRDFHMSVVRARRNDSG | CTGAACTGGTACAGAATGAGCCCCAGCAACCAGACCGAC |
| ICOS | TYLCGAISLAPKAQIKESLR | AAGCTGGCCGCCTTTCCTGAGGATAGATCTCAGCCCGGC |
| Intracellular | AELRVTERRAEVPTAHPSPS | CAGGACTGCCGGTTCAGAGTTACACAGCTGCCCAACGGC |
| Domain | PRPAGQFQTLVFWLPIGCAA | CGGGACTTCCACATGTCTGTCGTCCGGGCCAGAAGAAAC |
| | FVVVCILGCILICWLTKKKY | GACAGCGGCACATATCTGTGCGGCGCCATTTCTCTGGCC |
| | SSSVHDPNGEYMFMRAVNTA | CCTAAGGCTCAGATCAAAGAGAGCCTGAGAGCCGAGCTG |
| | KKSRLTDVTL (SEQ ID | AGAGTGACAGAAAGACGGGCCGAAGTGCCCACAGCTCAC |
| | NO: 43) | CCTTCACCTTCTCCAAGACCTGCCGGCCAGTTTCAGACC |
| | | CTGGTGTTCTGGCTGCCTATCGGCTGTGCCGCTTTTGTG |
| | | GTCGTGTGCATCCTGGGCTGCATCCTGATCTGCTGGCTG |
| | | ACCAAGAAAAAGTACAGCAGCAGCGTGCACGACCCCAAC |
| | | GGCGAGTACATGTTCATGAGAGCCGTGAACACCGCCAAG |
| | | AAGTCCAGACTGACCGACGTGACCCTT (SEQ ID NO: |
| | | 44) |

In some aspects, the embodiments relates to a nucleic acid molecule encoding a chimeric transmembrane protein as described herein. The nucleic acid molecule may comprise a promoter, wherein the promoter is operably linked to a nucleotide sequence encoding the chimeric transmembrane protein, e.g., for expression of a chimeric transmembrane protein in a recombinant cell. In some embodiments, the promoter is a constitutive promoter. In some embodiments, the promoter is a cell specific promoter. In some embodiments, the promoter is a tissue specific promoter.

The nucleic acid molecule may comprise the sequence set forth above. The nucleic acid molecule may comprise a nucleotide sequence having at least about 90%, 91%, 92%, 93%, 94%, 95%, 96%, 97%, 98%, or 99% sequence homology with the nucleotide sequence set forth herein. Since the genetic code is degenerate variations in the nucleic acid sequence may not change the encoded amino acid sequence. Accordingly, degenerate changes are intended to be encompassed by the present disclosure. In some embodiments, the nucleic acid molecule may comprise a nucleotide sequence having at least about 90%, 91%, 92%, 93%, 94%, 95%, 96%, 97%, 98%, or 99% sequence homology with at least about 100, 200, 300, 400, 500, 600, or 700 consecutive nucleotides in the nucleotide sequence set forth herein. For example, the nucleic acid molecule may comprise a nucleotide sequence having at least 95% sequence homology with at least 100 consecutive nucleotides in the nucleotide sequence set forth herein. Homology can be used running Blastn or BlastP at the NCBI website using default settings to compare or align two sequences.

In some embodiments, the nucleic acid molecule encodes an amino acid sequence as described herein. In some embodiments, the nucleic acid molecule encodes an amino acid sequence comprising one or more of the amino acid sequences set forth herein. In some embodiments, the nucleic acid molecule may comprise a nucleotide sequence that encodes an amino acid sequence having at least about 90%, 91%, 92%, 93%, 94%, 95%, 96%, 97%, 98%, or 99% sequence homology with an amino acid sequence set forth herein. Homology can be identity or similarly in the context of a protein. Homology can be used by employing routine tools such as Expasy, BLASTp, Clustal, and the like using default settings.

In some embodiments, the chimeric transmembrane protein comprises one or more amino acid sequences set forth herein and above.

In some embodiments, the chimeric transmembrane protein comprises an amino acid sequence having at least about 90%, 91%, 92%, 93%, 94%, 95%, 96%, 97%, 98%, or 99% sequence homology with one of the amino acid sequences set forth herein.

Variants of the amino acid sequences described herein may be included in various embodiments. The term “variant” refers to a protein or polypeptide in which one or more (e.g., 1, 2, 3, 4, etc.) amino acid substitutions, deletions, and/or insertions are present as compared to the amino acid sequence of a protein or polypeptide, and the term includes naturally occurring allelic variants and alternative splice variants of a protein or polypeptide. The term “variant” includes the replacement of one or more amino acids in an amino acid sequence with a similar or homologous amino acid(s) or a dissimilar amino acid(s). Some variants include alanine substitutions at one or more amino acid positions in an amino acid sequence. Other substitutions include conservative substitutions that have little or no effect on the overall net charge, polarity, or hydrophobicity of the protein. Conservative substitutions may have insignificant effect on the function of the chimeric transmembrane protein. In some embodiments, the function can be the specificity of a protein when expressed in a lymphocyte, e.g., a marrow-infiltrating lymphocyte (MIL). One of skill in the art can determine if a substitution affects the function of a chimeric transmembrane protein by comparing to the sequences provided herein. Non-limiting exemplary conservative substitutions are set forth in the table below. According to some embodiments, a chimeric transmembrane protein has at least 90%, 91%, 92%, 93%, 94%, 95%, 96%, 97%, 98%, or 99% sequence identity with an amino acid sequence described herein.

Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | proline |
| | methionine |
| | leucine |
| | isoleucine |

The table below sets out another scheme of conservative amino acid substitutions.

| Original Residue | Conservative Substitutions |
|---|---|
| Ala | Gly; Ser; Thr |
| Arg | Lys; Gln |
| Asn | Gln; His; Ser |
| Asp | Glu; Asn |
| Cys | Ser |
| Gln | Asn; Ser; Asp; Glu |
| Glu | Asp; Gln; Lys |
| Gly | Ala; Pro; Asn |
| His | Asn; Gln; Tyr |
| Ile | Leu; Val; Met; Val; Phe |
| Leu | Ile; Val; Met; Phe |
| Lys | Arg; Gln |
| Met | Leu; Tyr; Ile; Val; Phe |
| Pro | Ser; Thr; Ala; Gly |
| Phe | Met; Leu; Tyr; Trp |
| Ser | Thr; Gly; Asn; Asp |
| Thr | Ser; Asn |
| Trp | Tyr; Phe |
| Tyr | Trp; Phe |
| Val | Ile; Leu; Met; Phe |

Accordingly, in some embodiments, 1, 2, 3, 4, 5, 6, 7, 8, 9, or 10 of the amino acid residues of an amino acid sequence disclosed herein are modified with conservative substitutions. In some embodiments, only 1, 2, 3, 4 or 5 amino acid residues are substituted with conservative substitutions.

In some embodiments, the chimeric transmembrane protein comprises a sequence (SEQ ID NO: 1-44) described herein or a variant thereof. In some embodiments, if the protein comprises a leader sequence of CD8 (SEQ ID NO: 1) it is replaced with another signal peptide or leader sequence, that can assist in trafficking the chimeric transmembrane protein to the extracellular membrane.

In some aspects, the embodiments relate to a recombinant cell, comprising a nucleic acid as disclosed herein. In some embodiments, the embodiments relate to a recombinant cell, comprising a chimeric transmembrane protein as described herein. In some embodiments, the cell comprises a chimeric protein comprising an amino acid sequence set forth herein or a variant thereof. In some embodiments, the cell is a lymphocyte. The cell may be a T cell. In some embodiments, the cell may be a tumor-infiltrating lymphocyte (“TIL”) or a marrow infiltrating lymphocyte (“MIL”).

In some embodiments, the cell comprising a chimeric transmembrane protein described herein persist longer in a subject when administered to the subject as compared to a cell without a chimeric transmembrane protein.

In some aspects, the embodiments relate to a method for making a recombinant cell, comprising transfecting a cell with a nucleic acid molecule as described herein. In some aspects, the embodiments relate to a method for making a recombinant cell, comprising transfecting a cell with a nucleic acid molecule encoding an amino acid sequence as described herein. The nucleic acid molecule may be a plasmid. The cell can be transfected by a plasmid comprising one or more nucleotide sequences as described herein. The cell can also be infected with a virus or virus-like particle comprising the nucleic acid molecule. In some embodiments, the virus is a lentivirus, adenovirus, or adeno-associated virus (“AAV”). In some embodiments, the cell is a TIL or a MIL. In some embodiments, the MIL is an activated MIL. MILs can be activated, for example, by incubating them with anti-CD3/anti-CD28 beads and appropriate cytokines, e.g., under hypoxic conditions. An example of growing the MILs under hypoxic conditions can found, for example, in WO2016037054, which is hereby incorporated by reference in its entirety. In some embodiments, the nucleic acid molecule is transfected into a cell after the cell has been incubated in a hypoxic environment as described herein. In some embodiments, the nucleic acid molecule is transfected into a cell after the cell has been incubated in a hypoxic environment for about 1, 2, 3, 4, or 5 days. In some embodiments, the cell is then incubated under normoxic conditions for about 1, 2, 3, 4, or 5 days.

In some embodiments, a MIL comprising the chimeric transmembrane protein is prepared according to a method described in WO2016037054, which is hereby incorporated by reference in its entirety. In some embodiments, the method may comprise removing cells in the bone marrow, lymphocytes, and/or marrow infiltrating lymphocytes (“MILs”) from the subject; incubating the cells in a hypoxic environment, thereby producing activated MILs; and administering the activated MILs to the subject. The cells can also be activated in the presence of anti-CD3/anti-CD28 antibodies and cytokines as described herein. A nucleic acid molecule encoding a chimeric transmembrane protein, such as one of those described herein, can be transfected or infected into a cell before or after the MIL is incubated in a hypoxic environment.

The hypoxic environment may comprise less than about 21% oxygen, such as less than about 20%, 19%, 18%, 17%, 16%, 15%, 14%, 13%, 12%, 1 1%, 10%, 9%, 8%, 7%, 6%, 5%, 4%, or less than about 3% oxygen. For example, the hypoxic environment may comprise about 0% oxygen to about 20% oxygen, such as about 0% oxygen to about 19% oxygen, about 0% oxygen to about 18% oxygen, about 0% oxygen to about 17% oxygen, about 0% oxygen to about 16% oxygen, about 0% oxygen to about 15% oxygen, about 0% oxygen to about 14% oxygen, about 0% oxygen to about 13% oxygen, about 0% oxygen to about 12% oxygen, about 0% oxygen to about 11% oxygen, about 0% oxygen to about 10% oxygen, about 0% oxygen to about 9% oxygen, about 0% oxygen to about 8% oxygen, about 0% oxygen to about 7% oxygen, about 0% oxygen to about 6% oxygen, about 0% oxygen to about 5% oxygen, about 0% oxygen to about 4% oxygen, or about 0% oxygen to about 3% oxygen. In some embodiments, the hypoxic environment comprises about 1% to about 7% oxygen. In some embodiments, the hypoxic environment is about 1% to about 2% oxygen. In some embodiments, the hypoxic environment is about 0.5% to about 1.5% oxygen. In some embodiments, the hypoxic environment is about 0.5% to about 2% oxygen. The hypoxic environment may comprise about 20%, 19%, 18%, 17%, 16%, 15%, 14%, 13%, 12%, 1 1%, 10%, 9%, 8%, 7%, 6%, 5%, 4%, 3%, 2%, 1%, or about 0% oxygen. In some embodiments, the hypoxic environment comprises about 7%, 6%, 5%, 4%, 3%, 2%, or 1% oxygen.

Incubating MILs in a hypoxic environment may comprise incubating the MILs, e.g., in tissue culture medium, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 1 1 days, 12 days, 13 days, or even at least about 14 days. Incubating may comprise incubating the MILs for about 1 hour to about 30 days, such as about 1 day to about 20 days, about 1 day to about 14 days, or about 1 day to about 12 days. In some embodiments, incubating MILs in a hypoxic environment comprises incubating the MILs in a hypoxic environment for about 2 days to about 5 days. The method may comprise incubating MILs in a hypoxic environment for about 1 day, 2 days, 3 days, 4 days, 5 days, 6 days, 7 days, 8 day, 9 days, 10 days, 11 days, 12 days, 13 days, or 14 days. In some embodiments, the method comprises incubating the MILs in a hypoxic environment for about 3 days. In some embodiments, the method comprises incubating the MILs in a hypoxic environment for about 2 days to about 4 days. In some embodiments, the method comprises incubating the MILs in a hypoxic environment for about 3 days to about 4 days.

In some embodiments, the method further comprises incubating the MILs in a normoxic environment, e.g., after incubating the MILs in a hypoxic environment.

The normoxic environment may comprise at least about 21% oxygen. The normoxic environment may comprise about 5% oxygen to about 30% oxygen, such as about 10% oxygen to about 30% oxygen, about 15% oxygen to about 25% oxygen, about 18% oxygen to about 24% oxygen, about 19% oxygen to about 23% oxygen, or about 20% oxygen to about 22% oxygen. In some embodiments, the normoxic environment comprises about 21% oxygen.

Incubating MILs in a normoxic environment may comprise incubating the MILs, e.g., in tissue culture medium, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 1 1 days, 12 days, 13 days, or even at least about 14 days. Incubating may comprise incubating the MILs for about 1 hour to about 30 days, such as about 1 day to about 20 days, about 1 day to about 14 days, about 1 day to about 12 days, or about 2 days to about 12 days.

In some embodiments, the cell is transfected or infected with a nucleic acid molecule encoding a chimeric transmembrane protein described herein after being placed in a normoxic environment or before it is placed in a normoxic environment.

In some embodiments, the MILs are obtained by extracting a bone marrow sample from a subject and culturing/incubating the cells as described herein. In some embodiments, the bone marrow sample is centrifuged to remove red blood cells. In some embodiments, the bone marrow sample is not subject to apheresis. In some embodiments, the bone marrow sample does not comprise peripheral blood lymphocytes (“PBL”) or the bone marrow sample is substantially free of PBLs. These methods select for cells that are not the same as what have become to be known as TILs. Thus, a MIL is not a TIL. TILs can be selected by known methods to one of skill in the art and can be transfected or infected with the nucleic acid molecules described herein such that the TILs can express the chimeric transmembrane protein described herein.

In some embodiments, the cells are also activated by culturing with antibodies to CD3 and CD28. This can be performed, for example by incubating the cells with anti-CD3/anti-CD28 beads that are commercially available or that can be made by one of skill in the art. The cells can then be plated in a plate, flask, or bag. Hypoxic conditions can be achieved by flushing either the hypoxic chamber or cell culture bag for 3 minutes with a 95% Nitrogen and 5% CO₂ gas mixture. This can lead to, for example, 1-2% or less O₂ gas in the receptacle. Cells can be then cultured as described herein or as in the examples of WO2016037054, which is hereby incorporated by reference.

In some embodiments, a hypoxic MIL comprising a chimeric transmembrane protein as described herein is provided. In some embodiments, the hypoxic MIL is in an environment of about 0.5% to about 5% oxygen gas. In some embodiments, the hypoxic MIL is in an environment of about 1% to about 2% oxygen gas. In some embodiments, the hypoxic MIL is in an environment of about 1% to about 3% oxygen gas. In some embodiments, the hypoxic MIL is in an environment of about 1% to about 4% oxygen gas. A hypoxic MIL is a MIL that has been incubated in a hypoxic environment, such as those described herein, for a period of time, such as those described herein. Without being bound to any particular theory, a hypoxic MIL will undergo changes in protein and/or gene expression that affect the anti-tumor capabilities of the MIL. As described herein, the hypoxic MIL can also be activated with the presence of anti-CD3/anti-CD28 beads or other similar activating reagents. Thus, a hypoxic MIL can also be an activated-hypoxic MIL.

In some aspects, the embodiments relates to a method for increasing an immune response in a subject, comprising administering to the subject a recombinant cell as described herein. In some embodiments, the embodiments relate to a method for treating a neoplasm in a subject, comprising administering to the subject a recombinant cell as described herein. The neoplasm may be a benign neoplasm, a malignant neoplasm, or a secondary neoplasm. The neoplasm may be cancer. The neoplasm may be a lymphoma or a leukemia, such as chronic lymphocytic leukemia (“CLL”) or acute lymphoblastic leukemia (“ALL”). The neoplasm may be multiple myeloma as well as any solid tumor (e.g., breast cancer, prostate cancer, lung cancer, esophageal cancer, brain cancer, kidney cancer, bladder cancer, pancreatic cancer, osteosarcoma, and the like). The cancer can also be a cancer described herein.

The method may comprise administering to the subject a plurality of recombinant cells as described herein. The method may comprise administering to the subject an effective amount of recombinant cells as described herein. In some embodiments, the cell is an autologous cell with respect to the subject receiving the recombinant cells. In some embodiments, the cell is an allogenic cell with respect to the subject receiving the recombinant cells. In some embodiments, the cell is a xenogenic cell with respect to the subject receiving the recombinant cells.

Accordingly, in some embodiments, the cell is a cell obtained from the subject and modified with the receptor provided for herein and then administered back to the subject. The cell can be as described herein.

In some embodiments, the cell is a cell obtained from a different subject and modified with the receptor provided for herein and then administered back to a subject that is not the same as the source of the cells. The cell can be as described herein.

In some embodiments, the cell is a cell obtained from a different species (e.g. pig) and modified with the receptor provided for herein and then administered back to a subject that is not the same as the source of the cells. The cell can be as described herein.

In some embodiments, the cell is obtained from the subject. The cell that is transfected or infected may be obtained from the subject. The cell can be obtained as described herein. For example, a cell that is administered may be autologous to the subject. In some embodiments, the cell that is administered is allogeneic to the subject. The cell may be obtained from the subject and transfected or infected with a nucleic acid encoding a chimeric transmembrane protein as described herein. The cell may be a daughter cell, wherein a parent of the daughter cell was obtained from the subject. The recombinant cell may have been transfected or infected with the nucleic acid or a parent of the recombinant cell may have been transfected or infected with the nucleic acid. In some embodiments, the cell after being transfected or infected expresses a protein comprising one or more of the amino sequences described herein.

The method may further comprise making the recombinant cell, wherein making the recombinant cell comprises transfecting or infecting a cell with a nucleic acid encoding a chimeric transmembrane protein, such as those described herein. In some embodiments, the chimeric transmembrane protein comprises an amino acid sequence set forth in any one of SEQ ID NO: 5, 6, 7, 8, 9, 10, or 11 or a variant thereof. Similarly, the method may further comprise making a plurality of recombinant cells, wherein making the plurality of recombinant cells comprises transfecting or infecting a plurality of cells with nucleic acids encoding a chimeric transmembrane protein, such as those described herein. The method may further comprise expanding a parent cell, e.g., the recombinant cell may be a daughter cell of the parent cell. The method may comprise expanding a population of cells, e.g., the method may comprise administering to the subject a plurality of recombinant cells as described herein, and each cell of the plurality of recombinant cells may be a daughter cell of a parent cell.

The method may further comprise isolating the cell or a parent cell from the subject.

The method may further comprise sorting the cell, e.g., by fluorescence activated cell sorting (“FACS”) or magnetic activated cell sorting (“MACS”).

The cells can be administered to a subject by any suitable route in, for example, a pharmaceutically acceptable composition. In some embodiments, the composition is pyrogen free. For example, administration of the cells may be carried out using any method known in the art. For example, administration may be parenteral, intravenous, intra-arterial, subcutaneous, intramuscular, intracranial, intraorbital, ophthalmic, intraventricular, intracapsular, intraspinal, intracisternal, intraperitoneal, intracerebroventricular, or intrathecal. For parenteral administration, the cells may be administered by either intravenous, subcutaneous, or intramuscular injection, in compositions with pharmaceutically acceptable vehicles or carriers. The cells can be formulated for parenteral administration by injection, for example, by bolus injection or continuous infusion. The compositions can take such forms as suspensions, solutions, or emulsions in oily or aqueous vehicles, and can contain formulatory agents, for example, suspending, stabilizing, and/or dispersing agents.

For administration by injection, it can be desired to use the cells in solution in a sterile aqueous vehicle which may also contain other solutes such as buffers or preservatives as well as sufficient quantities of pharmaceutically acceptable salts or of glucose to make the solution isotonic. In some embodiments, the pharmaceutical compositions may be formulated with a pharmaceutically acceptable carrier to provide sterile solutions or suspensions for injectable administration. In particular, injectables can be prepared in conventional forms, either as liquid solutions or suspensions or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like. Suitable pharmaceutical carriers are described in “Remington's pharmaceutical Sciences” by E. W. Martin.

The subject may be any organism that comprises immune cells. For example, the subject may be selected from rodents, canines, felines, porcines, ovines, bovines, equines, and primates. The subject may be a mouse or a human.

In some embodiments, The subject may have a neoplasm. The neoplasm may be a benign neoplasm, a malignant neoplasm, or a secondary neoplasm. The neoplasm may be cancer. The neoplasm may be a lymphoma or a leukemia, such as chronic lymphocytic leukemia (“CLL”) or acute lymphoblastic leukemia (“ALL”). The subject may have a glioblastoma, medulloblastoma, breast cancer, head and neck cancer, kidney cancer, ovarian cancer, Kaposi's sarcoma, acute myelogenous leukemia, and B-lineage malignancies. The subject may have multiple myeloma.

In some embodiments, the subject is a subject “in need thereof” As used herein, the phrase “in need thereof” means that the subject has been identified or suspected as having a need for the particular method or treatment. In some embodiments, the identification can be by any means of diagnosis. In any of the methods and treatments described herein, the subject can be in need thereof.

The receptors provided herein can also placed into cells with other chimeric activated receptors, which can also be referred to as a “CAR”.

### EXAMPLES

The following examples are illustrative, but not limiting, of the methods and compositions described herein. Other suitable modifications and adaptations of the variety of conditions and parameters normally encountered in therapy and that are obvious to those skilled in the art are within the spirit and scope of the embodiments.

### Example 1

PD-1 switch receptor expression in lentivirus transduced Jurkat leukemia cell line. The receptors described below were transduced into Jurkat cells using a lentivirus expression system and as described herein. Briefly, Jurkat cells were transduced with lentivirus carrying an empty vector control carrying green fluorescent protein (GFP) only (Empty Vector) or with lentiviruses carrying each of the six PD-1 switch receptors linked to GFP by a T2a cleavable peptide. Four days following transduction, the cells were labelled with either anti-PD-PECy7 or with an isotype-matched control antibody and analyzed using a Beckman Coulter Galios flow cytometer. Untransduced Jurkat cells that do not express PD-1 PD-1 switch receptors or GFP (Untransduced) were labelled in the same way and used as a negative control. The receptors were found to be expressed in the cells.

Expression was also determined by binding the cells with tetrameric human PDL1 and analyzing by FACS, which demonstrated that the PD-1 switch receptors were capable of binding the PD-1 ligand PDL1. This was determined by using the following procedure. Briefly, Jurkat cells were transduced with lentivirus carrying an empty vector control carrying green fluorescent protein (GFP) only (Empty Vector) or with lentiviruses carrying each of the six PD-1 switch receptors linked to GFP by a T2a cleavable peptide. The cells were labelled with tetrameric human PDL1-Ig tagged with the fluorescent molecule phycoerythrin (PE) and analyzed using a Beckman Coulter Galios flow cytometer. Untransduced Jurkat cells that do not express PD-1, PD-1 switch receptors or GFP (Untransduced) were labelled in the same way and used as a negative control.

The receptors were also found to be expressed after being transduced into MILs. Briefly, Bone Marrow (BM) from three multiple myeloma patients (A) Patient 476-2312, B) Patient 1431, C) Patient 1943) were used to generate activated MILs under hypoxic conditions. On day 3, the MILs were transduced with lentivirus carrying an empty vector control carrying GFP only (Empty Vector) or with lentiviruses carrying each of the six PD1 switch receptors linked to GFP by a T2a cleavable peptide. Three days following transduction, the cells were labeled with anti-CD3-APC, anti-CD8-APCH7, Live-dead Yellow, and either anti-PD1-PECy7 or an isotype-matched control antibody, and analyzed using a Beckman Coulter Galios flow cytometer. Untransduced MILs that do not express the PD1 switch receptors or GFP (Untransduced) were labeled in the same way and used as a negative control. The expression was measured by flow cytometry. The increase in PD1-expression observed in GFP+PD-1 switch receptor-transduced MILs compared to empty vector control-transduced MILs corresponds to the expression of the PD1 switch receptors. The MILs had been activated under hypoxic conditions. These results demonstrate that the receptors were capable of being expressed in different cell types. Cells expressing the receptors can be used to treat neoplasms, such as the cancers described herein.

### Example 2: Hypoxic Activated MILs Treat Cancer

MILs obtained from subjects are activated and expanded as described herein. Briefly, after the marrow sample is obtained from the subject, the cells are incubated under hypoxic conditions in the presence of anti-CD3/-anti-CD28 beads and cytokines as described in WO2016037054, which is hereby incorporated by reference. The MILs are then infected with a virus comprising a nucleic acid molecule encoding a chimeric transmembrane protein comprising SEQ ID NO: 21, 23, 25, 27, 29, or 31. The nucleic acid molecule can also be introduced by transfection or transduction. The chimeric receptor may also comprise a leader sequence as provided herein. The cells are then grown under normoxic conditions and allowed to expand. The control and infected MILs are contacted with different cell types. Neither the expansion of the MILS nor the ability of the MILs to recognize antigens is negatively affected by the presence of the chimeric transmembrane protein. Adding a chimeric transmembrane protein to a MIL is not detrimental to its functions and growth. The MILs are administered to a subject with cancer, such as multiple myeloma, and the cancer is treated and the subject is in remission. The cells are also found to persist and continue to keep the subject in remission.

In summary, the embodiments and examples provided herein demonstrate that cells expressing a chimeric transmembrane protein provided herein can be effectively used to treat cancer and/or modulate an immune response.

Any U.S. patents, U.S. patent application publications, U.S. patent applications, foreign patents, foreign patent applications and non-patent publications, including CAS numbers, referred to in this specification and/or listed in the Application Data Sheet are incorporated herein by reference, in their entirety.

1. A protein comprising:
an extracellular domain of PD-1;
a transmembrane domain selected from the group consisting of: 4-1 BB transmembrane domain, CD28 transmembrane domain, CD27 transmembrane domain, and ICOS transmembrane domain; and
an intracellular signaling domain selected from the group consisting of 4-1 BB intracellular signaling domain, CD28 intracellular signaling domain, CD27 intracellular signaling domain, and ICOS intracellular signaling domain, and any combination thereof.

2. The protein of claim 1, wherein the transmembrane domain is the 4-1BB transmembrane domain.

3. The protein of claim 2, wherein the intracellular signaling domain is selected from the group consisting of: 4-1 BB intracellular signaling domain, CD28 intracellular signaling domain, CD27 intracellular signaling domain, and ICOS intracellular signaling domain.

4. The protein of claim 2, wherein the intracellular signaling domain is the intracellular domain of 4-1 BB and an intracellular domain selected from the group consisting of: CD28 intracellular signaling domain, CD27 intracellular signaling domain, and ICOS intracellular signaling domain.

5. The protein of claim 1, wherein the transmembrane domain is the CD28 transmembrane domain.

6. The protein of claim 5, wherein the intracellular signaling domain is selected from the group consisting of: 4-1 BB transmembrane domain, CD28 transmembrane domain, CD27 transmembrane domain, and ICOS transmembrane domain.

7. The protein of claim 5, wherein the intracellular signaling domain is the intracellular domain of 4-1 BB and an intracellular domain selected from the group consisting of: CD28 intracellular signaling domain, CD27 intracellular signaling domain, and ICOS intracellular signaling domain.

8. The protein of claim 1, wherein the transmembrane domain is the CD27 transmembrane domain.

9. The protein of claim 8, wherein the intracellular signaling domain is selected from the group consisting of: 4-1 BB intracellular signaling domain, CD28 intracellular signaling domain, CD27 intracellular signaling domain, and ICOS intracellular signaling domain.

10. The protein of claim 8, wherein the intracellular signaling domain is the intracellular domain of 4-1 BB and an intracellular domain selected from the group consisting of: CD28 intracellular signaling domain, CD27 intracellular signaling domain, and ICOS intracellular signaling domain.

11. The protein of claim 1, wherein the transmembrane domain is the ICOS transmembrane domain.

12. The protein of claim 11, wherein the intracellular signaling domain is selected from the group consisting of: 4-1 BB intracellular signaling domain, CD28 intracellular signaling domain, CD27 intracellular signaling domain, and ICOS intracellular signaling domain.

13. The protein of claim 11, wherein the intracellular signaling domain is the intracellular domain of 4-1 BB and the intracellular domain selected from the group consisting of: CD28 intracellular signaling domain, CD27 intracellular signaling domain, and ICOS intracellular signaling domain.

14. The protein of claim 1, wherein the protein comprises a sequence of SEQ ID NO: 21, 23, 25, 27, 29, or 31.

15. The protein of claim 1, wherein the protein comprises a leader sequence.

16. The protein of claim 15, wherein the protein comprises a CD8 leader sequence.

17. The protein of claim 16, wherein the CD8 leader sequence comprises SEQ ID NO: 1.

18. A nucleic acid molecule encoding the protein of claim 1.

19. A nucleic acid molecule comprising a sequence of SEQ ID NO: 22, 24, 26, 28, 30, or 32.

20. The nucleic acid molecule of claim 19, further comprising a leader sequence encoded by SEQ ID NO: 2.

21. A recombinant cell, comprising the nucleic acid molecule of claim 19.

22. A recombinant cell, comprising the nucleic acid molecule of claim 18.

23. A recombinant cell, comprising the protein of claim 1.

24. The cell of claim 21, wherein the cell is a lymphocyte or a T cell.

25. The cell of claim 21, wherein the cell is a tumor infiltrating lymphocyte (“TIL”).

26. The cell of claim 21, wherein the cell is a marrow infiltrating lymphocyte (“MIL”).

27. The cell of claim 26, wherein the MIL is a hypoxic MIL.

28. A method for making a recombinant cell, comprising transfecting or infecting a cell with a nucleic acid molecule encoding a protein of claim 1.

29. The method of claim 28, wherein the cell is a MIL.

30. The method of claim 28 further comprising incubating the MIL under hypoxic conditions prior to transfecting or infecting the cell with the nucleic acid molecule encoding the protein.

31. The method of claim 30, wherein the hypoxic conditions comprise about 0.5% to about 5% oxygen gas.

32. The method of claim 30, wherein the hypoxic conditions comprise about 1% to about 2% oxygen gas.

33. The method of claim 30, further comprising incubating the cells under normoxic conditions after the hypoxic incubation.

34. The method of claim 28, further comprising contacting the cell with anti-CD3/anti-CD28 beads.

35. A method for increasing an immune response in a subject, comprising administering to the subject the recombinant cell of claim 21.

36. The method of claim 35, further comprising making the recombinant cell, wherein making the recombinant cell comprises transfecting a cell with a nucleic acid encoding the protein.

37. The method of claim 35, further comprising isolating the cell from the subject.

38. The method of claim 35, wherein the subject has a neoplasm.

39. The method of claim 35, wherein the neoplasm is a leukemia, lymphoma, or multiple myeloma.

40. The method of claim 35, wherein the subject is a human.

41. A method for treating a neoplasm in a subject, comprising administering to the subject the recombinant cell of claim 21.

42. The method of claim 41, further comprising making the recombinant cell, wherein making the recombinant cell comprises transfecting a cell with a nucleic acid encoding the chimeric transmembrane protein.

43. The method of claim 41, further comprising isolating the cell from the subject.

44. The method of claim 41, wherein the subject has a neoplasm.

45. The method of claim 44, wherein the neoplasm is a multiple myeloma, leukemia or lymphoma.

46. The method of claim 41, wherein the subject is a human.

47. The method of claim 41, further comprising prior to administering the cell to the subject:
contacting the cell with anti-CD3/anti-CD28 beads;
incubating the cell under hypoxic conditions; and
incubating the cell under normoxic conditions.

48. The method of claim 47, wherein the cell is incubated under hypoxic conditions for about 0.5 to about 4 days.

49. The method of claim 47, wherein the cell is incubated under normoxic conditions for about 0.5 to about 4 days.


SEQUENCE LISTING (44 sequences)

SEQ ID NO: 1 - protein, 21 aa, Artificial Sequence
  CD Leader Peptide
MALPVTALLLPLALLLHAARP

SEQ ID NO: 2 - DNA, 63 bp, Artificial Sequence
  CD Leader Peptide
atggccctgcccgtgaccgccctgctcctgcctctggctctgctgctgcatgccgccagacct

SEQ ID NO: 3 - protein, 150 aa, Artificial Sequence [sequence omitted]

SEQ ID NO: 4 - DNA, 450 bp, Artificial Sequence
  PD-1 Extracellular Domain
cccggctggttcctggacagccccgacagaccctggaaccctcccaccttcagccctgccctgctcgtggtgacagagggcgacaacgccaccttcacctgtagcttcagcaacaccagcgagagcttcgtgctgaactggtacagaatgagccccagcaaccagaccgacaagctggccgccttccccgaggacagaagccagcccggccaggactgccggttcagagtgacccagctgcccaacggccgggacttccacatgagcgtggtgcgcgccagacggaacgacagcggcacatacctgtgcggcgccatcagcctggcccctaaggcccagatcaaagagagcctgcgggccgagctgagagtgaccgagagaagggccgaggtgcccaccgcccaccctagcccatctccaagacctgccggccagttccagaccctggtg

SEQ ID NO: 5 - protein, 27 aa, Artificial Sequence
  4-1BB Transmembrane Domain
IISFFLALTSTALLFLLFFLTLRFSVV

SEQ ID NO: 6 - DNA, 81 bp, Artificial Sequence
  4-1BB Transmembrane Domain
atcatctcattctttctggccctgaccagcacagccctgctgtttctgctgttcttcctgaccctgcggttcagcgtggtg

SEQ ID NO: 7 - protein, 42 aa, Artificial Sequence
  4-1BB Intracellular Domain
KRGRKKLLYIFKQPFMRPVQTTQEEDGCSCRFPEEEEGGCEL

SEQ ID NO: 8 - DNA, 51 bp, Artificial Sequence
  4-1BB Intracellular Domain
aaacggggcagaaagaagctgctgtacatcttcaagcagcccttcatgcgg

SEQ ID NO: 9 - protein, 27 aa, Artificial Sequence
  CD28 Transmembrane Domain
FWVLVVVGGVLACYSLLVTVAFIIFWV

SEQ ID NO: 10 - DNA, 81 bp, Artificial Sequence
  CD28 Transmembrane Domain
ttttgggtgctcgtggtcgttggcggagtgctggcctgttatagcctgctggtcaccgtggccttcatcatcttttgggtc

SEQ ID NO: 11 - protein, 41 aa, Artificial Sequence
  CD28 Intracellular Domain
RSKRSRLLHSDYMNMTPRRPGPTRKHYQPYAPPRDFAAYRS

SEQ ID NO: 12 - DNA, 123 bp, Artificial Sequence
  CD28 Intracellular Domain
cgaagcaagcggagccggctgctgcacagcgactacatgaacatgacccctagacggcccggacctaccagaaagcactaccagccttacgctcctcctagagacttcgccgcctacagatct

SEQ ID NO: 13 - protein, 21 aa, Artificial Sequence
  CD27 Transmembrane Domain
ILVIFSGMFLVFTLAGALFLH

SEQ ID NO: 14 - DNA, 63 bp, Artificial Sequence
  CD27 Transmembrane Domain
atcctcgtgatcttcagcggcatgttcctggtgttcacactggctggcgccctgtttctgcac

SEQ ID NO: 15 - protein, 48 aa, Artificial Sequence
  CD27 Intracellular Domain
QRRKYRSNKGESPVEPAEPCRYSCPREEEGSTIPIQEDYRKPEPACSP

SEQ ID NO: 16 - DNA, 144 bp, Artificial Sequence
  CD27 Intracellular Domain
cagcggagaaagtacagaagcaacaagggcgagagccccgtggaacctgccgagccttgtagatacagctgtcccagagaggaagagggcagcacaatccccatccaagaggactacagaaagcccgagcctgcctgctctcct

SEQ ID NO: 17 - protein, 21 aa, Artificial Sequence
  ICOS Transmembrane Domain
FWLPIGCAAFVVVCILGCILI

SEQ ID NO: 18 - DNA, 63 bp, Artificial Sequence
  ICOS Transmembrane Domain
ttctggctgcctatcggctgtgccgcttttgtggtcgtgtgcatcctgggctgcatcctgatc

SEQ ID NO: 19 - protein, 38 aa, Artificial Sequence
  ICOS Intracellular Domain
CWLTKKKYSSSVHDPNGEYMFMRAVNTAKKSRLTDVTL

SEQ ID NO: 20 - DNA, 114 bp, Artificial Sequence
  ICOS Intracellular Domain
tgctggctgaccaagaaaaagtacagcagcagcgtgcacgaccccaacggcgagtacatgttcatgagagccgtgaacaccgccaagaagtccagactgaccgacgtgaccctt

SEQ ID NO: 21 - protein, 219 aa, Artificial Sequence
  PD-1 Extracellular Domain; 4-1BB Transmembrane Domain; 4-1BB Intracellular Domain
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIISFFLALTSTALLFLLFFLTLRFSVVKRGRKKLLYIFKQPFMRPVQTTQEEDGCSCRFPEEEEGGCEL

SEQ ID NO: 22 - DNA, 657 bp, Artificial Sequence
  PD-1 Extracellular Domain; 4-1BB Transmembrane Domain; 4-1BB Intracellular Domain
cccggctggttcctggacagccccgacagaccctggaaccctcccaccttcagccctgccctgctcgtggtgacagagggcgacaacgccaccttcacctgtagcttcagcaacaccagcgagagcttcgtgctgaactggtacagaatgagccccagcaaccagaccgacaagctggccgccttccccgaggacagaagccagcccggccaggactgccggttcagagtgacccagctgcccaacggccgggacttccacatgagcgtggtgcgcgccagacggaacgacagcggcacatacctgtgcggcgccatcagcctggcccctaaggcccagatcaaagagagcctgcgggccgagctgagagtgaccgagagaagggccgaggtgcccaccgcccaccctagcccatctccaagacctgccggccagttccagaccctggtgatcatctcattctttctggccctgaccagcacagccctgctgtttctgctgttcttcctgaccctgcggttcagcgtggtgaaacggggcagaaagaagctgctgtacatcttcaagcagcccttcatgcggcccgtgcagaccacccaggaagaagacggctgcagctgccggttccccgaagaagaagagggcggctgcgaactg

SEQ ID NO: 23 - protein, 218 aa, Artificial Sequence
  PD-1 Extracellular Domain; CD28 Transmembrane Domain; CD28 Intracellular Domain
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVFWVLVVVGGVLACYSLLVTVAFIIFWVRSKRSRLLHSDYMNMTPRRPGPTRKHYQPYAPPRDFAAYRS

SEQ ID NO: 24 - DNA, 654 bp, Artificial Sequence
  PD-1 Extracellular Domain; CD28 Transmembrane Domain; CD28 Intracellular Domain
ccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtgttttgggtgctcgtggtcgttggcggagtgctggcctgttatagcctgctggtcaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatct

SEQ ID NO: 25 - protein, 260 aa, Artificial Sequence
  PD-1 Extracellular Domain; CD28 Transmembrane Domain; CD28 Intracellular Domain; 4-1BB Intracellular Domain
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 26 - DNA, 780 bp, Artificial Sequence
  PD-1 Extracellular Domain; CD28 Transmembrane Domain; CD28 Intracellular Domain; 4-1BB Intracellular Domain
ccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtgttttgggtgctcgtggtcgttggcggagtgctggcctgttatagcctgctggtcaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgcagattccccgaggaagaagaaggcggctgcgagctt

SEQ ID NO: 27 - protein, 219 aa, Artificial Sequence
  PD-1 Extracellular Domain; CD27 Transmembrane Domain; CD27 Intracellular Domain
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVILVIFSGMFLVFTLAGALFLHQRRKYRSNKGESPVEPAEPCRYSCPREEEGSTIPIQEDYRKPEPACSP

SEQ ID NO: 28 - DNA, 657 bp, Artificial Sequence
  PD-1 Extracellular Domain; CD27 Transmembrane Domain; CD27 Intracellular Domain
ccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtcatcctcgtgatcttcagcggcatgttcctggtgttcacactggctggcgccctgtttctgcaccagcggagaaagtacagaagcaacaagggcgagagccccgtggaacctgccgagccttgtagatacagctgtcccagagaggaagagggcagcacaatccccatccaagaggactacagaaagcccgagcctgcctgctctcct

SEQ ID NO: 29 - protein, 261 aa, Artificial Sequence
  PD-1 Extracellular Domain; CD27 Transmembrane Domain; CD27 Intracellular Domain; 4-1BB Intracellular Domain
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVILVIFSGMFLVFTLAGALFLHQRRKYRSNKGESPVEPAEPCRYSCPREEEGSTIPIQEDYRKPEPACSPKRGRKKLLYIFKQPFMRPVQTTQEEDGCSCRFPEEEEGGCEL

SEQ ID NO: 30 - DNA, 783 bp, Artificial Sequence
  PD-1 Extracellular Domain; CD27 Transmembrane Domain; CD27 Intracellular Domain; 4-1BB Intracellular Domain
ccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtcatcctcgtgatcttcagcggcatgttcctggtgttcacactggctggcgccctgtttctgcaccagcggagaaagtacagaagcaacaagggcgagagccccgtggaacctgccgagccttgtagatacagctgtcccagagaggaagagggcagcacaatccccatccaagaggactacagaaagcccgagcctgcctgctctcccaagagaggcagaaagaagctgctgtacatcttcaagcagcccttcatgcggcccgtgcagaccacacaagaggaagatggctgctcctgcagattccccgaggaagaagaaggcggctgcgagctt

SEQ ID NO: 31 - protein, 209 aa, Artificial Sequence
  PD-1 Extracellular Domain; ICOS Transmembrane Domain; ICOS Intracellular Domain
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVFWLPIGCAAFVVVCILGCILICWLTKKKYSSSVHDPNGEYMFMRAVNTAKKSRLTDVTL

SEQ ID NO: 32 - DNA, 627 bp, Artificial Sequence
  PD-1 Extracellular Domain; ICOS Transmembrane Domain; ICOS Intracellular Domain
ccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtgttctggctgcctatcggctgtgccgcttttgtggtcgtgtgcatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctt

SEQ ID NO: 33 - protein, 240 aa, Artificial Sequence
  PD-1 Extracellular Domain; 4-1BB Transmembrane Domain; 4-1BB Intracellular Domain
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIISFFLALTSTALLFLLFFLTLRFSVVKRGRKKLLYIFKQPFMRPVQTTQEEDGCSCRFPEEEEGGCEL

SEQ ID NO: 34 - DNA, 720 bp, Artificial Sequence
  PD-1 Extracellular Domain; 4-1BB Transmembrane Domain; 4-1BB Intracellular Domain
atggccctgcccgtgaccgccctgctcctgcctctggctctgctgctgcatgccgccagacctcccggctggttcctggacagccccgacagaccctggaaccctcccaccttcagccctgccctgctcgtggtgacagagggcgacaacgccaccttcacctgtagcttcagcaacaccagcgagagcttcgtgctgaactggtacagaatgagccccagcaaccagaccgacaagctggccgccttccccgaggacagaagccagcccggccaggactgccggttcagagtgacccagctgcccaacggccgggacttccacatgagcgtggtgcgcgccagacggaacgacagcggcacatacctgtgcggcgccatcagcctggcccctaaggcccagatcaaagagagcctgcgggccgagctgagagtgaccgagagaagggccgaggtgcccaccgcccaccctagcccatctccaagacctgccggccagttccagaccctggtgatcatctcattctttctggccctgaccagcacagccctgctgtttctgctgttcttcctgaccctgcggttcagcgtggtgaaacggggcagaaagaagctgctgtacatcttcaagcagcccttcatgcggcccgtgcagaccacccaggaagaagacggctgcagctgccggttccccgaagaagaagagggcggctgcgaactg

SEQ ID NO: 35 - protein, 239 aa, Artificial Sequence
  PD-1 Extracellular Domain; CD28 Transmembrane Domain; CD28 Intracellular Domain
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVFWVLVVVGGVLACYSLLVTVAFIIFWVRSKRSRLLHSDYMNMTPRRPGPTRKHYQPYAPPRDFAAYRS

SEQ ID NO: 36 - DNA, 717 bp, Artificial Sequence
  PD-1 Extracellular Domain; CD28 Transmembrane Domain; CD28 Intracellular Domain
atggccctgcctgtgacagctctgctgctgcctcttgctctgcttctgcatgccgctagacctccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtgttttgggtgctcgtggtcgttggcggagtgctggcctgttatagcctgctggtcaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatct

SEQ ID NO: 37 - protein, 281 aa, Artificial Sequence
  PD-1 Extracellular Domain; CD28 Transmembrane Domain; CD28 Intracellular Domain; 4-1BB Intracellular Domain
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 38 - DNA, 843 bp, Artificial Sequence
  PD-1 Extracellular Domain; CD28 Transmembrane Domain; CD28 Intracellular Domain; 4-1BB Intracellular Domain
atggccctgcctgtgacagctctgctgctgcctcttgctctgcttctgcatgccgctagacctccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtgttttgggtgctcgtggtcgttggcggagtgctggcctgttatagcctgctggtcaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgcagattccccgaggaagaagaaggcggctgcgagctt

SEQ ID NO: 39 - protein, 240 aa, Artificial Sequence
  PD-1 Extracellular Domain; CD27 Transmembrane Domain; CD27 Intracellular Domain
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVILVIFSGMFLVFTLAGALFLHQRRKYRSNKGESPVEPAEPCRYSCPREEEGSTIPIQEDYRKPEPACSP

SEQ ID NO: 40 - DNA, 720 bp, Artificial Sequence
  PD-1 Extracellular Domain; CD27 Transmembrane Domain; CD27 Intracellular Domain
atggccctgcctgtgacagctctgctgctgcctcttgctctgcttctgcatgccgctagacctccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtcatcctcgtgatcttcagcggcatgttcctggtgttcacactggctggcgccctgtttctgcaccagcggagaaagtacagaagcaacaagggcgagagccccgtggaacctgccgagccttgtagatacagctgtcccagagaggaagagggcagcacaatccccatccaagaggactacagaaagcccgagcctgcctgctctcct

SEQ ID NO: 41 - protein, 282 aa, Artificial Sequence
  PD-1 Extracellular Domain; CD27 Transmembrane Domain; CD27 Intracellular Domain; 4-1BB Intracellular Domain
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVILVIFSGMFLVFTLAGALFLHQRRKYRSNKGESPVEPAEPCRYSCPREEEGSTIPIQEDYRKPEPACSPKRGRKKLLYIFKQPFMRPVQTTQEEDGCSCRFPEEEEGGCEL

SEQ ID NO: 42 - DNA, 846 bp, Artificial Sequence
  PD-1 Extracellular Domain; CD27 Transmembrane Domain; CD27 Intracellular Domain; 4-1BB Intracellular Domain
atggccctgcctgtgacagctctgctgctgcctcttgctctgcttctgcatgccgctagacctccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtcatcctcgtgatcttcagcggcatgttcctggtgttcacactggctggcgccctgtttctgcaccagcggagaaagtacagaagcaacaagggcgagagccccgtggaacctgccgagccttgtagatacagctgtcccagagaggaagagggcagcacaatccccatccaagaggactacagaaagcccgagcctgcctgctctcccaagagaggcagaaagaagctgctgtacatcttcaagcagcccttcatgcggcccgtgcagaccacacaagaggaagatggctgctcctgcagattccccgaggaagaagaaggcggctgcgagctt

SEQ ID NO: 43 - protein, 230 aa, Artificial Sequence
  PD-1 Extracellular Domain; ICOS Transmembrane Domain; ICOS Intracellular Domain
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVFWLPIGCAAFVVVCILGCILICWLTKKKYSSSVHDPNGEYMFMRAVNTAKKSRLTDVTL

SEQ ID NO: 44 - DNA, 690 bp, Artificial Sequence
  PD-1 Extracellular Domain; ICOS Transmembrane Domain; ICOS Intracellular Domain
atggccctgcctgtgacagctctgctgctgcctcttgctctgcttctgcatgccgctagacctccaggctggttcctggactctcccgacagaccttggaaccctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagaccctggtgttctggctgcctatcggctgtgccgcttttgtggtcgtgtgcatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctt